(19) United States
(12) Patent Application Publication
(10) Pub. No.: US 2022/0022872 A1
(43) Pub. Date: Jan. 27, 2022

(54) REMOTELY ADJUSTABLE MECHANISM AND ASSOCIATED SYSTEMS AND METHODS

(71) Applicant: W. L. Gore & Associates, Inc., Newark, DE (US)

(72) Inventors: Edward H. Cully, Newark, DE (US); Jeffrey B. Duncan, Flagstaff, AZ (US); Christine M. Scotti, Newark, DE (US)

(21) Appl. No.: 17/427,383

(22) PCT Filed: Feb. 1, 2019
(86) PCT No.: PCT/US2019/016345

§ 371 Date: Jul. 30, 2021

(51) Int. Cl. A61B 17/11

(52) U.S. Cl. CPC A61B 17/11; A61B 2017/00876; A61B 2017/1132; A61B 2017/1107

(57) ABSTRACT

Various aspects of the present disclosure are directed toward apparatuses, systems, and methods that include a medical device including a remotely actuated cross-sectional adjustment mechanism. The medical device may include a cross-sectional adjustment mechanism configured to actuate an implantable medical device between a first dimension and a second dimension that is larger than a first dimension to adjust an amount of fluid flow through the implantable medical device.

### CROSS-REFERENCE TO RELATED APPLICATION

This application is a U.S. 371 Application of International Application PCT/US2019/016345, filed Feb. 1, 2019, which is herein incorporated by reference in its entirety.

### FIELD

The present disclosure relates generally to implantable medical devices, and more specifically to mechanisms for remotely adjusting the cross-section and flow characteristics of implantable medical devices and associated systems and methods thereof.

### BACKGROUND

Implantable medical devices such as stents, stent-grafts, valves, and other intraluminal devices are used in a variety of medical procedures associated with various body passageways or body lumens to maintain, prevent, and/or adjust fluid flow therethrough. Such devices may be implanted in various locations within the body including in the vascular system, coronary system, respiratory system, urinary tract, and biliary tract, among others.

In some instances, the requisite size of the medical device may change over time. Current practices often require replacement of the device with a new, differently sized device altogether, which may require further operation and/or invasive procedures, causing added risk, stress and discomfort to the patient. In other instances, such as during the case of hemodialysis, the device might be oversized to permit treatment with less than optimal performance when a patient is not undergoing treatment (e.g., such as encouraging excess blood flow to the patient's heart).

### SUMMARY

In one example (“Example 1”), a medical device including a remotely actuated cross-sectional adjustment mechanism includes an implantable medical device defining a lumen; and a cross-sectional adjustment mechanism coupled to the implantable medical device, the cross-sectional adjustment mechanism configured to actuate the implantable medical device between a first dimension and a second dimension that is larger than a first dimension to adjust an amount of fluid flow through the lumen when an external adjustment force is applied to the cross-sectional adjustment mechanism.

In another example (“Example 2”), a medical device configured to be transcutaneously, cross-sectionally adjusted includes an implantable medical device defining a lumen; and a cross-sectional adjustment mechanism coupled to the implantable medical device, the cross-sectional adjustment mechanism configured to selectively actuate the implantable medical device from a first dimension to a second dimension that is larger than the first dimension by applying an external adjustment force on the cross-sectional adjustment mechanism in a first radial direction and from the second dimension to the first dimension by applying an external adjustment force on the cross-sectional adjustment mechanism in a second radial dimension that is different from the first radial direction.

In another example (“Example 3”), further to the medical device of Example 2, the cross-sectional adjustment mechanism includes a shape memory material configured to move between the first dimension and the second dimension in response to the external adjustment force.

In one example (“Example 4”), a medical device configured to be transcutaneously, cross-sectionally adjusted includes an implantable medical device defining a lumen; and a cross-sectional adjustment mechanism coupled to the implantable medical device, the cross-sectional adjustment mechanism configured to selectively actuate the implantable medical device between a first dimension towards a second dimension that is larger than the first dimension by applying heat in excess of body temperature on the cross-sectional adjustment mechanism and from the second dimension to the first dimension upon return to a temperature at or less than body temperature.

In another example (“Example 5”), further to the medical device of Example 4, the cross-sectional adjustment mechanism includes a heat adjustable material configured to maintain the first dimension when the implantable medical device is at body temperature and to maintain the second dimension when the implantable medical device is heated above body temperature.

In one example (“Example 6”), a medical device including a transcutaneously actuatable cross-sectional adjustment mechanism includes an implantable medical device defining a lumen; and a cross-sectional adjustment mechanism coupled to the implantable medical device, the cross-sectional adjustment mechanism configured to transition to a reduced dimension upon application of an external adjustment force and to maintain the reduced dimension upon removal of the external adjustment force.

In another example (“Example 7”), further to the medical device of Example 6, the cross sectional adjustment mechanism operates to maintain the reduced dimension via a magnetic force.

In one example (“Example 8”), a system for controlled blood flow includes an implantable medical device defining a lumen; and a cross-sectional adjustment mechanism coupled to the implantable medical device, the cross-sectional adjustment mechanism configured to transition to a reduced dimension upon application of an external adjustment force and to maintain the reduced dimension upon removal of the external adjustment force; and an external force applicator configured to apply the external adjustment force to the cross-sectional adjustment mechanism through the skin of a patient.

In another example (“Example 9”), further to the system of Example 8, the external force applicator is configured to apply a magnetic force to the cross-sectional adjustment mechanism.

In another example (“Example 10”), further to the system of any one of Examples 8-9, the cross-sectional adjustment mechanism is configured to lessen thrombus formation or stenosis in response to transitioning of the implantable medical device between the reduced dimension and a nominal diameter.

In another example (“Example 11”) a medical device configured to be actuated through the skin of a patient includes an implantable medical device defining a lumen and a wall, the wall including an outer surface and an inner surface; and a cross-sectional adjustment mechanism including a reservoir and a pocket between the outer surface and the inner surface of the wall, the reservoir being filled with fluid, the cross-sectional adjustment mechanism configured to actuate the implantable medical device between a first dimension and a second dimension that is larger than the first dimension to adjust an amount of fluid flow through the lumen when an external adjustment force is applied to the reservoir to transfer fluid from the reservoir into the pocket.

In another example (“Example 11”), further to the medical device of Example 10, the implantable medical device moves from the first dimension to the second dimension when fluid is removed from the pocket.

In one example (“Example 12”), a method for adjusting the cross-section of the medical device of any one of the preceding claims includes actuating the cross-sectional adjustment mechanism to move the implantable medical device from the first dimension to the second dimension; and actuating the cross-sectional adjustment mechanism to move the implantable medical device from the second dimension to the first dimension.

In another example (“Example 13”), further to the method of Example 12, the cross-sectional adjustment mechanism is actuated through the skin of a patient.

In another example (“Example 14”), further to the method of any one of Examples 12-13, the medical device forms an anastomosis between two vessels of a patient, the method further comprising accessing the medical device through the skin of a patient to access the anastomosis when the implantable medical device is at the first dimension, and actuating the cross-sectional adjustment mechanism to the second dimension after accessing the medical device through the skin of the patient to reduce flow through the implantable medical device.

In another example (“Example 15”), further to the method of any one of Examples 12-14, the method also includes coupling the cross-sectional adjustment mechanism to the implantable medical device, wherein the implantable medical device is one of a denovo graft or the implantable medical device that has been previously implanted.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 shows an implantable medical device for controlled blood flow implanted within a body of a patient, in accordance with an embodiment;

FIG. 2A is an end view of an implantable medical device including a remotely actuated diametric adjustment mechanism, in accordance with an embodiment;

FIG. 2B is a side view of an implantable medical device including a remotely actuated diametric adjustment mechanism, in accordance with an embodiment;

FIG. 2C is an end view of an implantable medical device including a remotely actuated diametric adjustment mechanism, in accordance with an embodiment;

FIG. 2D is a side view of an implantable medical device including a remotely actuated diametric adjustment mechanism, in accordance with an embodiment;

FIGS. 3A-3B is a side view of an implantable medical device, in accordance with an embodiment;

FIG. 4A is a side view of a medical device including a remotely actuated diametric adjustment mechanism, in accordance with an embodiment;

FIGS. 4B-4C are end views of a medical device including a remotely actuated diametric adjustment mechanism, in accordance with an embodiment;

FIGS. 5A-5B are side views of a medical device including a remotely actuated diametric adjustment mechanism, in accordance with an embodiment.

### DETAILED DESCRIPTION

Various aspects of the present disclosure relate to adjustment mechanisms for remotely adjusting cross-sectional areas of implantable medical devices. Examples of implantable medical devices can include stents, stent-grafts, valves, and devices for occlusion and/or anastomosis, among others. In certain examples, the implantable medical devices may be configured to adjust (e.g., increase and/or decrease) the size of a particular artificial or natural body lumen, passageway, and/or conduit to promote, restrict, or otherwise adjust fluid flow therethrough. Such diametric or sizing adjustment may be utilized as a precursor to a treatment, during a treatment, or following a treatment as desired.

For reference, the term “lumen” should be read broadly to include any of a variety of passages, such as those associated with the vasculature, biliary tract, urinary tract, lymph system, reproductive system, gastrointestinal system, or others. And, the term “cross-sectional area,” “diameter” or “diametric” should not be read to require a circular cross-section. Instead, such terms should be read to mean an effective cross-sectional area or diameter or in some cases a maximum transverse dimension of a cross-section. Thus, the term “cross-sectional area,” “diameter” or “diametric” can be read to mean more generally “size” unless specified otherwise.

In certain instances, it may be beneficial to temporarily adjust the diameter or cross-sectional area of implantable medical devices after implantation inside a patient's body. For example, in certain medical procedures (e.g., in the case of hemodialysis), higher blood flow through the device may be necessary or desired when performing the procedure. However, it may be beneficial to reduce blood flow through the device following the procedure so as not to provide an overflow of blood to the patient's heart.

In the above examples, it may also be beneficial to be able to adjust the implantable medical devices without additional, invasive procedures. Invasive procedures such as these can impart added stress and discomfort on the patient. Moreover, non-invasive procedures are generally less burdensome on health care providers in terms of preparation, treatment, and follow up efforts. In sum, various embodiments address solutions for achieving superior health benefits with reduced potential for additional burden on the patient and/or medical provider.

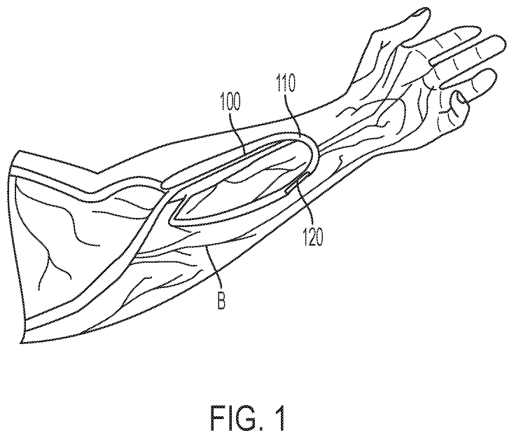

FIG. 1 shows an implantable medical device 100 for controlled blood flow implanted within a body B of a patient, in accordance with an embodiment. In the example of FIG. 1, the patient has been provided with an AV fistula, which is a connection of an artery to a vein and as shown in FIG. 1, placed in the forearm of the patient. Although an AV fistula is used as a primary example, it should be understood that any of a variety of body lumens and conduits are contemplated in association with the inventive concepts described herein.

The medical device 100 has been placed in the AV fistula and includes a conduit 110 and an adjustment mechanism 120 coupled to the conduit 110. In various examples, the conduit 110 defines an inner flow lumen and is configured to convey bodily fluid (e.g., blood) as desired. Although various forms are contemplated for the conduit 110, some examples include a graft, a stent graft, a heart valve, a vascular filter, or other implantable, medical device. Advantageous conduit forms may include grafts, stent grafts, heart valves, vascular filters or other implantable device conduits leveraging expanded polytetrafluoroethylene (ePTFE) technology, such as those products available from W.L. Gore and Associates, Inc. Any of a variety of additional and alternative materials are contemplated, and ePTFE examples should not be read in a limiting sense.

The adjustment mechanism (e.g., diametric) 120 is configured to actuate between a first dimension and a second dimension (e.g., which may be preset cross-sectional area(s) or diameter(s)) via application of an external adjustment mechanism (e.g., physical force, energy force, ultrasonic force, thermal force, or combinations thereof) through the skin of the patient without puncturing the skin of the patient (e.g., without creating a wound or other percutaneous access). The adjustment mechanism 120, in turn, may adjust a flow area of the conduit 110, and thus the device 100 between diameters or cross-sectional areas. In this way, the size or dimension of the lumen of the device 100, and thus flow through the device 100, may be adjusted externally (e.g., via application of the external adjustment force), reducing or eliminating the need for invasive procedures to replace the device 100 with another device having a different flow cross-sectional area or by otherwise modifying the flow cross-sectional area of the device 100 using invasive techniques.

As referenced above, in some instances, the device 100 forms an anastomosis between two vessels of the patient. The device 100 may be accessed through the patient's skin to adjust or modify flow through the anastomosis. For example, the adjustment mechanism 120 can be actuated to cause the device 100 to transition from a first cross-sectional area A1 to a second cross-sectional area A2 to enable a change in flow (e.g., an increase flow through the device 100). In some examples, the adjustment mechanism 120 may subsequently be actuated back to the first cross-sectional area A1 (e.g., to decrease flow through the device 100). In other examples, the adjustment mechanism 120 self-actuates, or self-deploys, without intervention by a user back to the first cross-sectional area A1 (e.g., after a period of time has passed).

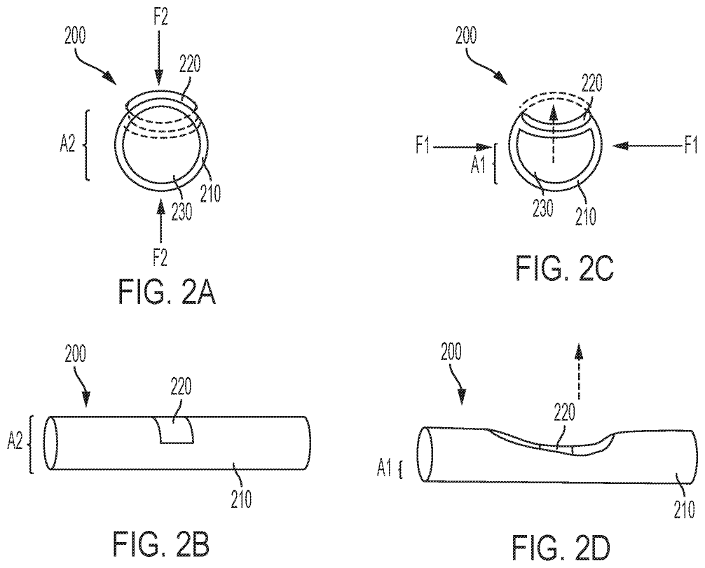

FIGS. 2A to 2D are end views and side views of an implantable medical device 200 including a conduit 210 having an inner flow lumen 230 and a diametric adjustment mechanism 220, in accordance with an embodiment. The implantable medical device 200 is optionally utilized as, or for, a portion of the implantable medical device 100 of FIG. 1 (e.g., as an anastomosis between two vessels) and incorporates any of the features described in association with the medical device 100 previously described. As shown, the diametric adjustment mechanism 220 is coupled to the conduit 210. In some instances, the diametric adjustment mechanism 220 is generally arranged around a portion of the outer surface of the conduit 210 such that a change in diameter of the diametric adjustment mechanism 220 also changes the cross-sectional area the inner flow lumen 230 of the conduit 210, and thus the inner flow lumen of the device 200.

In one example, the diametric adjustment mechanism 220 is configured to selectively actuate the inner flow lumen of the conduit 210, and more generally the device 200, between a first cross-sectional area A1 and a second cross-sectional area A2 that is larger than the first cross-sectional area A1. For example, in some instances, the first cross-sectional area A1 may be from about 2 mm to about 4 mm while the second cross-sectional area A2 may be from about 6 mm to about 8 mm depending on the desired flow dynamics through the lumen 230. Adjusting the device 200 between the first and second cross-sectional areas A1, A2 adjusts the amount of fluid flowing through the lumen 230, for example, from a smaller flow to a larger flow and vice versa. In other examples, there may be actuation between at least two different dimensions.

The cross-sectional adjustment mechanism 220 may be arranged on any portion of the conduit 210 as desired. In general, the length of the cross-sectional adjustment mechanism 220 is less than the total length of the device 200. For example, the length of the cross-sectional adjustment mechanism 220 may be from 80-90% the length of the device 200, from 70-80% the length of the device 200, from 50-70% the length of the device 200, or less than 50% the length of the device 200 as desired. For example, in some instances, the cross-sectional adjustment mechanism 220 may simply include a ring or cuff-like structure coupled to the outer surface of the conduit 210. In other examples, the cross-sectional adjustment mechanism 220 may be a stent-like structure overlying a portion of the conduit 210 or arranged within the lumen 230 of the conduit 210. In some examples, the adjustment mechanism may be along an inner surface of the conduit 210.

In the example of FIGS. 2A to 2D, the inner flow lumen 230 of the device 200 is adjusted between the first and second cross-sectional areas A1, A2 by applying an external adjustment force to the cross-sectional adjustment mechanism 220. For reference, FIGS. 2A and 2C are transverse cross-sections of the device 200 at the cross-sectional adjustment mechanism 220. FIGS. 2A and 2B show the medical device 200 transitioned to the second, larger cross-sectional area A2 and FIGS. 2C and 2D show the device transitioned to the first, smaller cross-sectional area A1.

The external adjustment force can be a physical, radial force (e.g., pinching) of the medical device 200 and, in particular, the cross-sectional adjustment mechanism 220. In particular, the cross-sectional adjustment mechanism 220 is configured such that a radial force in a first direction F1 (FIGS. 2C and 2D) causes the cross-sectional adjustment mechanism to expand, or flip outwardly, and in the second direction F2 (FIGS. 2A and 2B) causes the cross-sectional adjustment mechanism 220 to reversibly flip, or collapse inwardly to the state shown in FIGS. 2C and 2D. The collapsed or “pinched” configuration is maintained due to a resilience of the material and vice versa.

In this pinched or deflected position, the medical device 210 defines the first (smaller) cross-sectional area A1 with a smaller inner flow lumen 230. The adjustment mechanism 220 can be reversibly expanded by applying a physical, radial force (e.g., pinching) the adjustment mechanism 220 in a second direction (e.g., orthogonal to the first direction) to cause the cross-sectional adjustment mechanism to expand such that the medical device defines the second (larger) cross-sectional area A2 with the larger flow lumen 230.

For example, the cross-sectional adjustment mechanism 220 may be configured to move from the first cross-sectional area A1 to the second cross-sectional area A2 in response to an external adjustment force applied in a first radial direction F1 and may be configured to move from the second cross-sectional area A2 back to the first cross-sectional area A1 in response to an external adjustment force applied in a second radial direction F2 that is different than the first radial direction F1.

In some instances, the cross-sectional adjustment mechanism 220 may include an elastic or superelastic material (e.g., shape memory alloy) configured to transition between the first and second cross-sectional areas A1, A2 when the external adjustment force is applied to the diametric adjustment mechanism 220. In various examples, the cross-sectional adjustment mechanism 220 everts, or flips in orientation between the two diametric conditions, or cross-sectional areas A1 and A2.

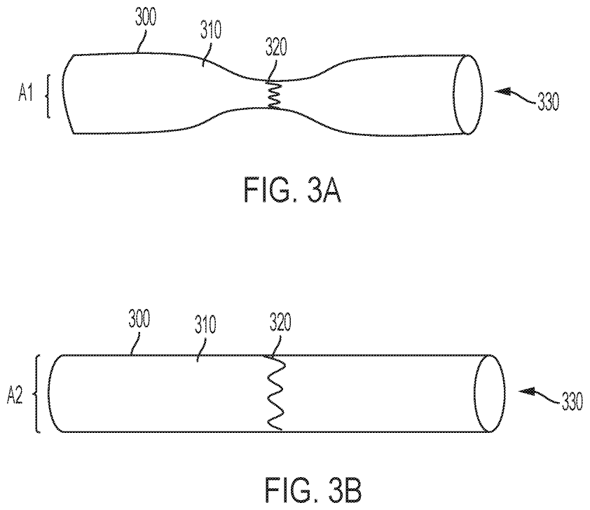

FIGS. 3A and 3B are side views of an implantable medical device 300 including a conduit 310 and a diametric adjustment mechanism 320, in accordance with an embodiment. As shown, the diametric adjustment mechanism 320 is coupled to the conduit 310 of the device 300. In some instances, the cross-sectional adjustment mechanism 320 is generally arranged around a portion of the outer surface of the conduit 310 such that a change in cross-sectional area of the cross-sectional adjustment mechanism 320 also changes the cross-sectional area of the inner flow lumen 330 of the conduit 310, and thus of the device 300.

The cross-sectional adjustment mechanism 320 is configured to selectively actuate the inner flow lumen 330 of the device 300 between a first cross-sectional area A1 and a second cross-sectional area A2 that is larger than the first cross-sectional area A1. For example, in some instances, the first cross-sectional area A1 may be from about 2 mm to about 4 mm while the second cross-sectional area A2 may be from about 6 mm to about 8 mm depending on desired flow dynamics through the inner flow lumen 330. Adjusting the cross-sectional adjustment mechanism 320 and thus the inner flow lumen 330 between the first and second cross-sectional areas A1, A2 adjusts the amount of fluid flowing through the inner flow lumen 330, for example, from a smaller flow to a larger flow and vice versa.

The cross-sectional adjustment mechanism 320 may be arranged on any portion of the conduit 310. In some instances, the diametric adjustment mechanism 320 may be approximately the same length as the overall length of the device 300, while in other instances, the length of the diametric adjustment mechanism 320 may be less than that of the overall length of the device 300. For example, the length of the diametric adjustment mechanism 320 may be from 80-90% the length of the device 300, from 70-80% the length of the device 300, from 50-70% the length of the device 300, or less than 50% the length of the device 300 as desired. For example, in some instances, the cross-sectional adjustment mechanism 320 may simply include a ring or cuff-like structure coupled to the outer surface of the device 300. In other examples, the cross-sectional adjustment mechanism 320 may be a stent-like structure overlying a portion of the device 300.

The device 300 is adjusted between the first and second cross-sectional areas A1, A2 by applying an external adjustment force to the cross-sectional adjustment mechanism 320. The external adjustment force can be any of a variety of types of forces. In some instances, the cross-sectional adjustment mechanism 320 may include a heat adjustable or thermally adjustable material configured to maintain the first cross-sectional area A1 when the device 300 is at body temperature, for example, and to maintain the second cross-sectional area A2 when the device 300 is heated above body temperature. For example, the cross-sectional adjustment mechanism 320 is optionally formed of a shape memory material, such as a nickel-titanium alloy that is configured to change phases above body temperature.

In some examples, upon application of an external heat source (e.g., a heat lamp, not shown), the shape memory material changes phases and causes the cross-sectional adjustment mechanism 300, and thus the inner flow lumen 330 of the conduit 310 and device 300 to transition from the first cross-sectional area A1 to the second cross-sectional area A2. Upon removal of the external heat source, the inner flow lumen of the device 300 returns from the second cross-sectional area A2 to the first cross-sectional area A1.

In other examples, thermal energy is applied to the device in the form of cooling (e.g., an ice pack) which causes the shape memory material to change phases and transition the inner flow lumen 330 of the device 300 from the first cross-sectional area A1 to the second cross-sectional area A2. Upon removal of the thermal energy (e.g., ice pack), the body of the patient warms causing the diametric adjustment mechanism 320 and thus the inner flow lumen 330 of the conduit 310 and device 300 to transition from the second cross-sectional area A2 to the first cross-sectional area A1.

Examples of suitable heat adjustable materials include any of a variety of shape memory materials, including nickel-titanium alloy shape memory materials (e.g., nitinol) among others, including polymeric shape memory or phase change materials

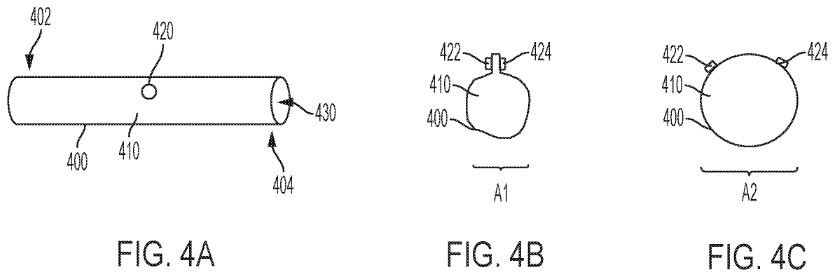

FIG. 4A is a side view of an implantable medical device 400 including a conduit 410 and a cross-sectional adjustment mechanism 420, in accordance with an embodiment. FIGS. 4B and 4C are end views of the implantable medical device 400 of FIG. 4A including the cross-sectional adjustment mechanism 420, in accordance with an embodiment. As shown in FIG. 4A, the cross-sectional adjustment mechanism 420 is coupled to the conduit 410. In general, the cross-sectional adjustment mechanism 420 may be coupled to the conduit 410 at any location along the conduit 410 (e.g., from a first end 402 to a second end 404 of the device 400).

The cross-sectional adjustment mechanism 420 is configured to selectively actuate the device 400 between a first cross-section A1 and a second cross-section A2 that is larger than the first cross-section A1, as shown in FIGS. 4B and 4C and in particular to adjust the inner flow lumen 430 of the conduit 410 between the first and second cross-sections A1 and A2. For example, in some instances, the first cross-section A1 may be from about 2 mm to about 4 mm while the second cross-section A2 may be from about 6 mm to about 8 mm depending on desired flow dynamics through the lumen 410. Adjusting the device 400, and in particular the inner flow lumen 430, between the first and second cross-sections A1, A2 adjusts the amount of fluid flowing through the inner flow lumen 430, for example, from a smaller flow to a larger flow and vice versa.

As shown in FIGS. 4B and 4C, in some instances, the cross-sectional adjustment mechanism 420 may include a first adjustment portion 422 and a second adjustment portion 424 arranged a distance from one another along the circumference of the conduit 410. The first and second adjustment portions 422, 424 are generally configured to mate with one another to pinch the device 400 and maintain the inner flow lumen 430 of the conduit 410, and more generally the device 400, at the first cross-section A1. The first and second adjustment portions 422, 424 are configured to separate from one another and move to the inn flower lumen 430 to the second cross-section A2 upon application of an external adjustment force. For example, in some instances, the cross-sectional adjustment mechanism 420 may include magnets configured to interact with one another to maintain the inner flow lumen 430 of the device 400 at the first cross-section A1 and configured to separate from one another upon application of a magnetic force to actuate the device 400 such that the inner flow lumen 430 transitions to the second cross-section A2 and vice versa.

In some instances, the external adjustment force may be applied with an external force applicator (not shown). For example, the external force applicator may be a magnet powerful enough to overcome the attraction between the first and second adjustment portions 422, 424 to separate the first and second adjustment portions 422, 424 from one another.

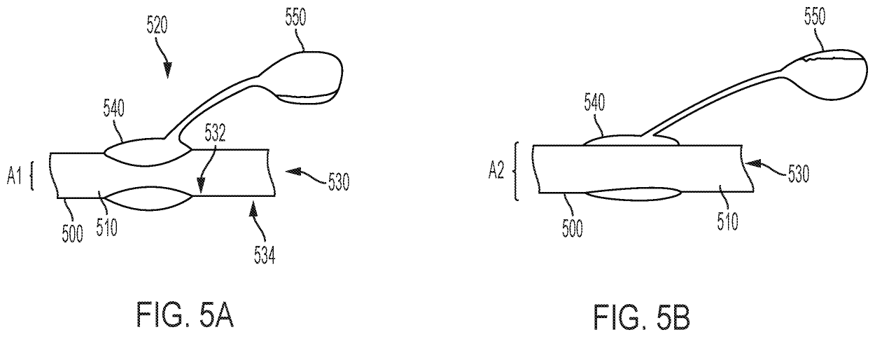

FIGS. 5A and 5B are side views of an implantable medical device 500 including a conduit 510 and a cross-sectional adjustment mechanism 520, in accordance with an embodiment. As shown, the conduit 510, and thus the device 500, defines an inner flow lumen 530 and includes a wall having an inner surface 532 and an outer surface 534. The cross-sectional adjustment mechanism 520 includes a pocket 540 located between the inner and outer surfaces 532, 534 of the wall. The pocket 540 is fluidly connected to a reservoir 550 configured to hold fluid (e.g., water, saline, or any other suitable fluid). Upon application of an external adjustment force to the cross-sectional adjustment mechanism 520, the fluid can be moved from the reservoir 550 to the pocket 540 to maintain the cross-section of the inner flow lumen 530 at a first cross-section A1 and from the pocket 540 back to the reservoir 550 to maintain the inner flow lumen 530 of the device 500 at a second cross-section A2.

The cross-sectional adjustment mechanism 520 is configured to selectively actuate the inner flow lumen 530 of the device 500 between the first cross-section A1 and a second cross-section A2 that is larger than the first cross-section A1. For example, in some instances, the first cross-section A1 may be from about 2 mm to about 4 mm while the second cross-section A2 may be from about 6 mm to about 8 mm depending on desired fluid dynamics through the lumen. Adjusting the device 500 between the first and second cross-sections A1, A2 adjusts the amount of fluid flowing through the inner flow lumen 530, for example, from a smaller flow to a larger flow and vice versa.

In general, fluid is maintained in the pocket 540 until the external adjustment force is applied to the pocket 540. In some instances, the external adjustment force is a force applied through the skin of the patient by a physician or other operator (e.g., to squeeze or pinch a portion of the cross-sectional adjustment mechanism 520). For example, the external adjustment force is applied to the pocket 540 to force the fluid from the pocket 540 into the reservoir 550 and actuate the inner flow lumen 530, and more generally the device 500 from the first cross-section A1 to the second cross-section A2. Similarly, the external adjustment force can be applied to the reservoir 550 to force fluid from the reservoir 550 back into the pocket 540 to actuate the inner flow lumen 530, and more generally the device 500 from the second cross-section A2 back to the first cross-section A1.

In some instances, the fluid can be a high-viscosity substance, such as a biocompatible polymer or gel material, for example, or a shear thinning fluid. Shear thinning materials such as hydrogels may be used. In this case, topical force applied will cause the viscous fluid to thin and flow. When the force causing shearing ceases, the material quickly returns to its viscous gel-like state until another force is applied. Magnetic liquids or “ferrofluids” are also contemplated. These materials change shape in the presence of a magnetic field. In these instances, the external adjustment force may also include heat that is applied through the patient's skin. The heat may reduce the viscosity of the fluid, allowing the fluid to flow between the pocket 540 and the reservoir 550. Once the heat is removed, the viscosity of the fluid increases, maintaining the fluid in the pocket 540 or the reservoir 550 and subsequently maintaining the device 500, and more specifically the inner flow lumen 530, at the first cross-section A1 or the second cross-section A2, respectively.

The cross-sectional area A1 may equal zero. Actuation of the device discussed herein, such as devices 200, 300, 400, 500 may be between fully open (A2) and completely closed (A1). Thus, when the devices 200, 300, 400, 500 are fully closed, blood will not flow. To prevent blood from clotting in these instances, a “locking solution” may be injected through one of the dialysis access needles. Locking solution, or Heparin lock is used as an anticoagulant in the dual lumen of Central Venous Catheters used in dialysis. The same solution could be used to prevent coagulation in the closed fistula until the next dialysis session triggers the cross-sectional change and the dialysis process begins again. In addition, actuation of the devices 200, 300, 400, 500, and more specifically a cross-sectional adjustment mechanism, may lessen the opportunity for thrombus formation or stenosis based on the transitioning of the implantable medical devices 200, 300, 400, 500 between the reduced dimension and a nominal diameter. The adjustment may lessen the opportunity for blood to stagnate, in certain instances, if transitioned on a daily or weekly basis.

The devices 200, 300, 400, 500 may be a separate device that is added to a prosthetic graft or native fistula or the devices 200, 300, 400, 500 may be manufactured as an integral component part of the graft. The cross-sectional adjustment mechanism (e.g., a remotely adjustable mechanism) may be added to a denovo graft, or may be added later after the graft has been implanted or fistula has been created. The implantation may be surgical or, in some embodiments, endovascular. In some instances, the remotely adjustable mechanism may be placed within the lumen of a device, while in other instances, the remotely adjustable mechanism is designed to be placed abluminally. It is conceived that combinations of these devices may be used together. For instance, it may be desirable to shut down an AV access graft at both ends and Heparin lock the segment between the remotely adjustable mechanisms.

Various methods of implantation the foregoing devices 100, 200, 300, 400, 500 include implanting the respective device at a site that is transcutaneously accessible without significant trauma (e.g., needle punctures) in order to actuate an associated cross-sectional adjustment device to modify a size of the inner flow lumen of the device. Persons skilled in the art will readily appreciate that various aspects of the present disclosure can be realized by any number of methods and apparatus configured to perform the intended functions. It should also be noted that the accompanying drawing figures referred to herein are not necessarily drawn to scale but may be exaggerated to illustrate various aspects of the present disclosure, and, in that regard, the drawing figures should not be construed as limiting.

1. A medical device including a remotely actuated cross-sectional adjustment mechanism, the device comprising:
an implantable medical device defining a lumen; and
a cross-sectional adjustment mechanism coupled to the implantable medical device, the cross-sectional adjustment mechanism configured to actuate the implantable medical device between a first dimension and a second dimension that is larger than a first dimension to adjust an amount of fluid flow through the lumen when an external adjustment force is applied to the cross-sectional adjustment mechanism.

2. A medical device configured to be transcutaneously, cross-sectionally adjusted, the device comprising:
an implantable medical device defining a lumen; and
a cross-sectional adjustment mechanism coupled to the implantable medical device, the cross-sectional adjustment mechanism configured to selectively actuate the implantable medical device from a first dimension to a second dimension that is larger than the first dimension by applying an external adjustment force on the cross-sectional adjustment mechanism in a first radial direction and from the second dimension to the first dimension by applying an external adjustment force on the cross-sectional adjustment mechanism in a second radial dimension that is different from the first radial direction.

3. The medical device of claim 2, wherein the cross-sectional adjustment mechanism includes a shape memory material configured to move between the first dimension and the second dimension in response to the external adjustment force.

4. A medical device configured to be transcutaneously, cross-sectionally adjusted, the device comprising:
an implantable medical device defining a lumen; and
a cross-sectional adjustment mechanism coupled to the implantable medical device, the cross-sectional adjustment mechanism configured to selectively actuate the implantable medical device between a first dimension towards a second dimension that is larger than the first dimension by applying heat in excess of body temperature on the cross-sectional adjustment mechanism and from the second dimension to the first dimension upon return to a temperature at or less than body temperature.

5. The medical device of claim 4, wherein the cross-sectional adjustment mechanism includes a heat adjustable material configured to maintain the first dimension when the implantable medical device is at body temperature and to maintain the second dimension when the implantable medical device is heated above body temperature.

6. A medical device including a transcutaneously actuatable cross-sectional adjustment mechanism, the device comprising:
an implantable medical device defining a lumen; and
a cross-sectional adjustment mechanism coupled to the implantable medical device, the cross-sectional adjustment mechanism configured to transition to a reduced dimension upon application of an external adjustment force and to maintain the reduced dimension upon removal of the external adjustment force.

7. The medical device of claim 6, wherein the cross sectional adjustment mechanism operates to maintain the reduced dimension via a magnetic force.

8. A system for controlled blood flow, the system comprising:
an implantable medical device defining a lumen; and
a cross-sectional adjustment mechanism coupled to the implantable medical device, the cross-sectional adjustment mechanism configured to transition to a reduced dimension upon application of an external adjustment force and to maintain the reduced dimension upon removal of the external adjustment force; and
an external force applicator configured to apply the external adjustment force to the cross-sectional adjustment mechanism through the skin of a patient.

9. The system of claim 8, wherein the external force applicator is configured to apply a magnetic force to the cross-sectional adjustment mechanism.

10. The system of claim 8, wherein the cross-sectional adjustment mechanism is configured to lessen thrombus formation or stenosis in response to transitioning of the implantable medical device between the reduced dimension and a nominal diameter.

11. A medical device configured to be actuated through the skin of a patient, the device comprising:
an implantable medical device defining a lumen and a wall, the wall including an outer surface and an inner surface; and
a cross-sectional adjustment mechanism including a reservoir and a pocket between the outer surface and the inner surface of the wall, the reservoir being filled with fluid, the cross-sectional adjustment mechanism configured to actuate the implantable medical device between a first dimension and a second dimension that is larger than the first dimension to adjust an amount of fluid flow through the lumen when an external adjustment force is applied to the reservoir to transfer fluid from the reservoir into the pocket.

12. The medical device of claim 11, wherein the implantable medical device moves from the first dimension to the second dimension when fluid is removed from the pocket.

13. A method for adjusting the cross-section of the medical device of any one of the preceding claims, the method comprising:
actuating the cross-sectional adjustment mechanism to move the implantable medical device from the first dimension to the second dimension; and
actuating the cross-sectional adjustment mechanism to move the implantable medical device from the second dimension to the first dimension.

14. The method of claim 13, wherein the cross-sectional adjustment mechanism is actuated through the skin of a patient.

15. The method of claim 13, wherein the medical device forms an anastomosis between two vessels of a patient, the method further comprising accessing the medical device through the skin of a patient to access the anastomosis when the implantable medical device is at the first dimension, and actuating the cross-sectional adjustment mechanism to the second dimension after accessing the medical device through the skin of the patient to reduce flow through the implantable medical device.

16. The method of claim 13, further comprising coupling the cross-sectional adjustment mechanism the implantable medical device, wherein the implantable medical device is one of a denovo graft or the implantable medical device has been previously implanted.
